(19) United States
(12) Patent Application Publication
(10) Pub. No.: US 2025/0306235 A1
(43) Pub. Date: Oct. 2, 2025

(54) METHODS AND SYSTEMS FOR SOURCE ROCK CHEMOSTRATIGRAPHY AND ORGANOFACIES CHARACTERIZATION

(71) Applicant: SAUDI ARABIAN OIL COMPANY, Dhahran (SA)

(72) Inventors: Pan Luo, Dhahran (SA); Guido Port, Dhahran (SA)

(73) Assignee: SAUDI ARABIAN OIL COMPANY, Dhahran (SA)

(21) Appl. No.: 18/622,371

(22) Filed: Mar. 29, 2024

(51) Int. Cl. G01V 9/00; E21B 49/02; G01N 1/08; G01N 30/12; G01N 30/68; G01N 33/24; G16C 20/20

(52) U.S. Cl. CPC G01V 9/005; E21B 49/02; G01N 1/08; G01N 30/12; G01N 33/241; E21B 2200/20; G01N 2030/125; G01N 30/68; G16C 20/20

(57) ABSTRACT

Methods and systems are disclosed. The method may include obtaining a first plurality of hydrocarbon source rock samples from a first well and a second plurality of hydrocarbon source rock samples from a second well, each well penetrating a portion of a subterranean region. For each of the first and second plurality of samples obtaining a set of kinetic parameter values, including a discrete distribution of activation energy and a common frequency factor and determining a weighted average activation energy value from the distribution. The method may further include identifying a first chemostratigraphic segment of the first well and a second chemostratigraphic segment of the second well, each based on the weighted average activation values; determining a correlated stratigraphic unit and mapping organofacies based on the first the second chemostratigraphic segments; and predicting hydrocarbon generation, retention and expulsion and determining a drilling target within the correlated stratigraphic unit.

### BACKGROUND

Source rock is defined as rock rich in organic matter that may generate, or may have generated, hydrocarbons when sufficiently heated. The generated hydrocarbons may be stored in the source rock or have been expelled from the source rock and migrated to a reservoir rock to be stored. As burial depth, heat, and time increase, the source rock may continue to generate hydrocarbons and the previously-generated hydrocarbons may thermally mature. As such, a subterranean region may store hydrocarbons of various thermal maturities in various locations.

Chemostratigraphy may be defined as the characterization of rock strata based on the geochemical composition of sediments, rocks, and constituents thereof and the correlation of rock strata across a subterranean region. Correlated rock strata at different locations have similar chemical composition. Similarly, organofacies are rock strata that share a collection of kerogens derived from common organic precursors, deposited under similar environments, and exposed to similar early diagenetic histories.

The rate of chemical reactions and their relation to temperature are frequently termed “kinetics”. For example, kinetics may describe the rate of conversion of kerogen to hydrocarbons under thermal stress. Kinetic parameters are important parameters used to characterize a source rock and a critical input to determine the rate of the conversion of kerogen to hydrocarbons over geological history.

However, due to the complex format of kinetic parameters, they have not been utilized in source rock chemostratigraphy and organofacies characterization.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method. The method may include obtaining, using a rock coring system, a first plurality of hydrocarbon source rock samples from a first well and a second plurality of hydrocarbon source rock samples from a second well, wherein the first well and the second well both penetrate a portion of a subterranean region. For each of the first plurality of hydrocarbon source rock samples and the second plurality of hydrocarbon source rock samples, using a pyrolysis system, the method includes obtaining a set of kinetic parameter values, where the set includes a discrete distribution of activation energy and a common frequency factor, and determining a weighted average activation energy value from the distribution. The method may further include, using a well log interpretation system, identifying a first chemostratigraphic segment of the first well based on the weighted average activation values of the first plurality of hydrocarbon source rock samples, where identifying the first chemostratigraphic segment includes determining a chemostratigraphic marker spanning a first range in depth, identifying a second chemostratigraphic segment of the second well based on the weighted average activation values of the second plurality of hydrocarbon source rock samples, wherein identifying the second chemostratigraphic segment includes determining a second range in depth, and determining a correlated stratigraphic unit based, at least in part, on the first chemostratigraphic segment and the second chemostratigraphic segment, wherein the correlated stratigraphic unit spans the portion of the subterranean region. The method may still further include determining a drilling target within the correlated stratigraphic unit.

In general, in one aspect, embodiments relate to a system including a rock coring system, a pyrolysis system, and a well log interpretation system. The rock coring system may be configured to obtain a first plurality of hydrocarbon source rock samples from a first well and a second plurality of hydrocarbon source rock samples from a second well, wherein the first well and the second well both penetrate a portion of a subterranean region. The pyrolysis system may be configured, for each of the first plurality of hydrocarbon source rock samples and the second plurality of rock samples, to obtain a set of kinetic parameter values, wherein the set includes a discrete distribution of activation energy and a common frequency factor, and determine a weighted average activation energy kinetic parameter value from the distribution. The well log interpretation system may be configured to identify a first chemostratigraphic segment of the first well based on the weighted average activation values of the first plurality of hydrocarbon source rock samples, wherein identifying the first chemostratigraphic segment includes determining a chemostratigraphic marker spanning a first range in depth and identify a second chemostratigraphic segment of the second well based on the weighted average activation values of the second plurality of hydrocarbon source rock samples, wherein identifying the second chemostratigraphic segment comprises determining a second range in depth. The well log interpretation system may be further configured to determine a correlated stratigraphic unit based, at least in part, on the first chemostratigraphic segment and the second chemostratigraphic segment, wherein the correlated stratigraphic unit spans the portion of the subterranean region, and determine a drilling target within the correlated stratigraphic unit.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 displays an organofacies classification system in accordance with one or more embodiments.

FIG. 2 illustrates a rock coring system in accordance with one or more embodiments.

FIG. 3 illustrates a pyrolysis system in accordance with one or more embodiments.

FIG. 4A displays pyrolysis data under different heating rates in accordance with one or more embodiments.

FIG. 4B displays absolute reaction rates versus temperature for a variety of heating rates derived from pyrolysis data in accordance with one or more embodiments.

FIG. 5 displays a cross-plot of weighted activation energy versus a logarithm occurrence frequency in accordance with one or more embodiments.

FIG. 6 displays weighted activation energy versus vitrinite reflectance for three wells in accordance with one or more embodiments.

FIG. 7 displays weighted activation energy versus depth for three wells in accordance with one or more embodiments.

FIG. 8 displays logs of maturity-corrected kinetic parameter values in accordance with one or more embodiments.

FIG. 9 shows a flowchart in accordance with one or more embodiments.

FIG. 10 shows a flowchart in accordance with one or more embodiments.

FIG. 11 illustrates a computer system in accordance with one or more embodiments.

FIG. 12 illustrates a drilling system in accordance with one or more embodiments.

FIG. 13 shows a systems flowchart in accordance with one or more embodiments.

### DETAILED DESCRIPTION

In the following detailed description of embodiments of the disclosure, numerous specific details are set forth to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a kinetic parameter” includes reference to one or more of such parameters.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

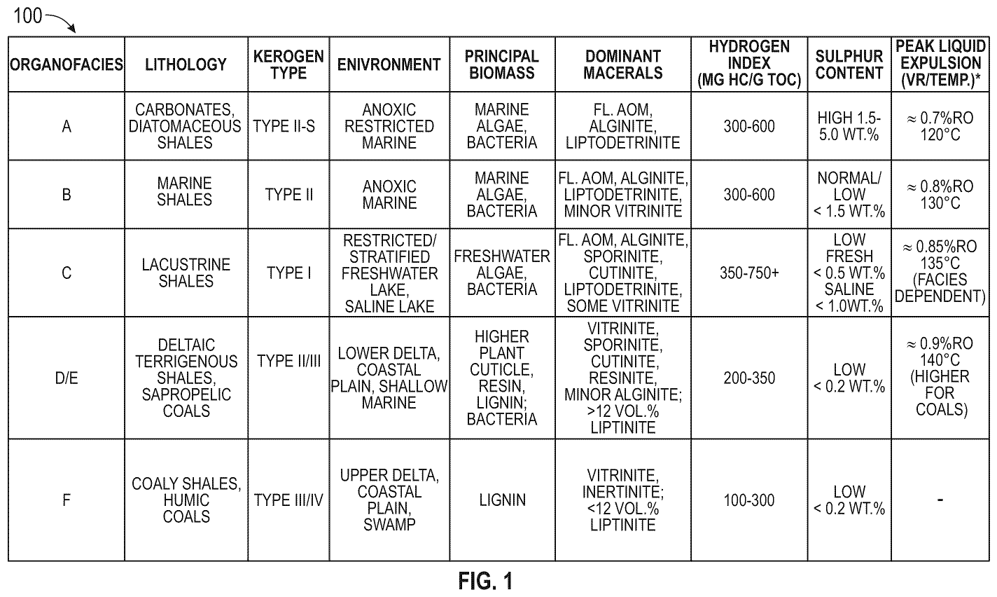

In the following description of FIGS. 1-13, any component described regarding a figure, in various embodiments disclosed herein, may be equivalent to one or more like-named components described regarding any other figure. For brevity, descriptions of these components will not be repeated regarding each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described regarding a corresponding like-named component in any other figure.

A stratum (i.e., rock body) within a subterranean region may be or include source rock and/or reservoir rock. Source rock may be defined as rock rich in organic matter where the organic matter may generate, or may have generated, hydrocarbons from kerogen when sufficiently heated. As burial depth, heat, and time increase, hydrocarbons may be generated from the kerogen in the source rock stratum. Kinetic parameters may be used to describe the conversation rate from kerogen to hydrocarbons. For example, a kinetic parameter may be an activation energy. Source rock may also be unconventional reservoir rock, serving as both the source and the reservoir of the hydrocarbon.

Each collection of kerogens derived from organic precursors within the source rock stratum may form or be categorized as a unique organofacies. Organofacies may categorize kerogen content, which may be heterogeneous, by kerogen type, common organic precursors, depositional environment (hereinafter “environment”), early diagenetic histories, etc. FIG. 1 displays an organofacies classification system 100 in accordance with one or more embodiments. The organofacies classification system 100 may include organofacies A, B, C, D/E, and F. Each organofacies may characterize kerogen relative to lithology, keorgen type, environment, principal biomass, dominant macerals, hydrogen index, sulphur content, and peak liquid expulsion (hereinafter collectively “characterization measures”). However, a person of ordinary skill in the art will appreciate that the organofacies classification system 100 may include other and/or additional characterizations and that the descriptions, values, and ranges provided in FIG. 1 are not absolute but a general guideline for what each of the organofacies A, B, C, D/E, and F may refer to.

However, such an organofacies classification system 100 may be of limited utility for petroleum system modeling. Petroleum system modeling may describe the process of modeling the evolution of a subterranean region over time to be used for prediction of unsampled spatial regions within a sedimentary basin. As such, a petroleum system model may model the generation, retention, expulsion, migration, and accumulation of hydrocarbons (e.g., petroleum) within the subterranean region over time. The petroleum system model may include other models that model specific processes and characteristics of features within the subterranean region. For example, the petroleum system model may include one or more source rock models, stratigraphy models, depositional models, compaction models, subsidence models, maturation models, migration models, etc. as well as the geometry of each feature within the subterranean region.

Returning to organofacies, the organofacies classification system 100 may be of limited utility within petroleum system modeling as the organofacies classification system 100 may only broadly or generally characterize kerogen. Use of such a broad characterization within a petroleum system model may inadequately represent the heterogeneities of organic matter in the source rock. For example, the petroleum system modeling based on the five organofacies shown in FIG. 1 and their published kinetics cannot accurately simulate the generation of hydrocarbon from the source rock in the subterranean region.

Chemostratigraphy is defined as the correlation and characterization of strata based on the geochemical composition of sediments, rock, and constituents thereof that make up the strata. Geochemical composition data and associated parameters used for the correlation and characterization of the strata may refer to the elemental/mineral contents, isotope ratios, chemical markers, or any proxy with geological interpretation derived from chemical analysis on the rocks. Chemostratigraphy is a branch of stratigraphy, a basic practice in geology, to study the age, characteristics, distribution and sequence of strata, and elucidate Earth history. Chemostratigraphy may be used to develop a detailed source rock model by correlation of source rock units and mapping of organofacies with in a petroleum system model for improving the simulation of hydrocarbon generation, retention, and expulsion in source rock. The invention describes a theme to use kinetic parameters for source rock chemostratigraphy and organofacies characterization.

The kinetics analysis (hereinafter simply “kinetics”) may include performing chemical analysis of reactants and products in a series of reactions (e.g., pyrolysis analysis) to determine reaction rates and their kinetic parameters. The parameters with proper assumption and optimization may be used to describe the rate of chemical reactions beyond the temperature range of experimental condition. Further, kinetics may include extrapolation values determined from laboratory data to conditions experienced in subterranean regions and geological time. In the context of this disclosure, kinetics may describe the chemical reaction rate of kerogen to hydrocarbons relative to temperature when the stratum that contains the kerogen is under thermal stress. Types of kinetics include bulk kinetics, oil and gas kinetics, and compositional kinetics. Bulk kinetics may refer to the process of converting kerogen to any hydrocarbon. Oil and gas kinetics may refer to the process of converting kerogen to oil and gas (i.e., specific hydrocarbons). Compositional kinetics may refer to the process of converting kerogen to specific hydrocarbon constituents, which may be based, at least in part, on carbon number (e.g., C₁, C₂, C₃-C₅, C₆-C₁₄ aromatics, C₆-C₁₄ saturates, C₁₄+ aromatics, and C₁₄+ saturates).

Kinetics may deploy an Arrhenius-type model. The Arrhenius-type model may quantitatively describe the conversion rate of kerogen to hydrocarbons as a series of irreversible reactions controlled by first-order chemical kinetics. First-order chemical kinetics may assume that the reaction rate k depends on the concentration of only one reactant and is proportional to the amount of the reactant. The Arrhenius-type model may take the form:

$$k = {Ae}^{- {E}_{a} / RT} , \qquad Equation ( 1 )$$

where k is the reaction rate that describes the change in molar mass of the reactant in units of time, A is the pre-exponential frequency factor that describes the number of potential elementary reactions per unit of time, Eₐ is the activation energy in kilocalories per mole (kcal/mol), R is the universal gas constant that is approximately 8.3145 Joules per mole times Kelvin (J/mol·K), T is the reaction temperature in Kelvin, and e is the base of the natural logarithm. The variables A and Eₐ may be considered as a set of kinetic parameters. In the context of this disclosure, the activation energy Eₐ may be the energy barrier that controls the rate at which kerogen is converted into hydrocarbons.

Equation (1) predicts the reaction rate at a given reaction temperature T, i.e., the temperature to which the reacting sample is exposed. Typically, the temperature to which a buried sediment is exposed may vary over geological time. For example, the temperature may increase over geological time due to increased burial depth of the stratum that contains the kerogen and the typical increase of temperature with depth found within the earth. Note in some cases, the temperature to which buried sediment is exposed may fluctuate as the depth of burial fluctuations due to cycles of deposition and erosion of the stack of strata overlying it.

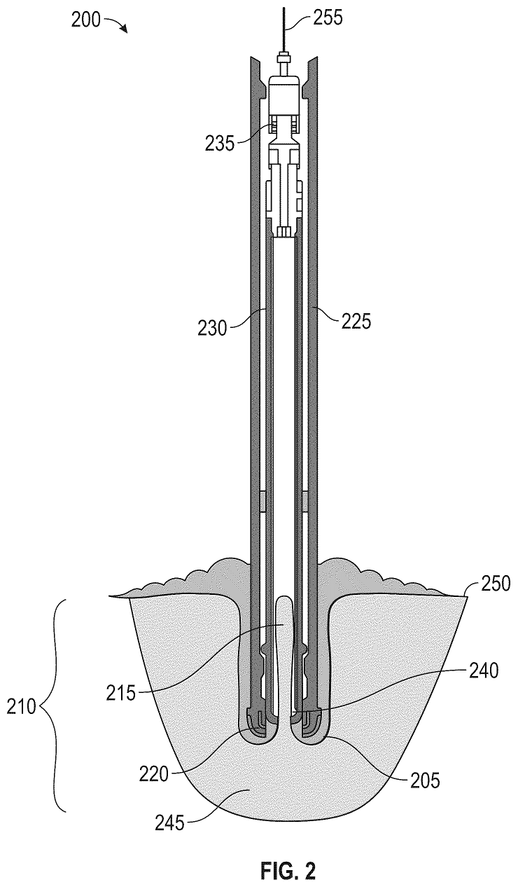

A distribution of a kinetic parameter may be determined by pyrolysis testing rock samples in a laboratory setting. The rock samples may be obtained from a subterranean region using a rock coring system. FIG. 2 illustrates a rock coring system 200 in accordance with one or more embodiments. The rock coring system 200 may be configured to simultaneously drill a well 205 within a subterranean region 210 and retrieve one or more rock cores 215 along intervals of the well 205. As such, the rock coring system 200 may be considered part of a drilling system. The rock coring system 200 may collect rock cores 215 continuously or at intervals while drilling the well 205. To do so, the rock coring system 200 may include a coring bit 220 attached to a core barrel 225. Within the core barrel 225, an inner barrel 230 may be disposed between a swivel 235 attached to an upper portion of the core barrel 225 and a core catcher 240 disposed close to the coring bit 220. The coring bit 220 consists of an annular cutting or grinding surface configured to flake, gouge, grind, or wear away strata 245 within the subterranean region 210 at the base or “toe” of the well 205. A central axial orifice may be configured to allow a cylindrical rock core 215 to pass through. The annular cutting surface of the coring bit 220 typically includes embedded polycrystalline compact diamond (PDC) cutting elements.

The inner barrel 230 within the core barrel 225 may be disposed above or behind the coring bit 220. Further, the inner barrel 230 may be separated from the coring bit 220 by the core catcher 240. As the coring bit 220 grinds away the strata 245 within the subterranean region 210, the cylindrical rock core 215 passes through the central orifice of the coring bit 220 and through the core catcher 240 into the inner barrel 230 as the coring bit 220 advances deeper into the subterranean region 210. The inner barrel 230 may be attached by the swivel 235 to the remainder of the core barrel 225 to permit the inner barrel 230 to remain stationary as the core barrel 225 rotates together with the coring bit 220. When the inner barrel 230 is filled with the rock core 215, the core barrel 225 containing the rock core 215 may be raised and retrieved at the surface of the earth 250. The core catcher 240 serves to grip the bottom of the rock core 215 and, as lifting tension is applied to the drillstring 255 and the core barrel 225, the rock core 215 breaks away from the undrilled strata 245 within the subterranean region 210 below the rock core 215. The core catcher 240 may retain the rock core 215 so that the rock core 215 does not fall out the bottom of the core barrel 225 through the annular orifice of the coring bit 220 as the core barrel 225 is raised to the surface of the earth 250.

In addition to collecting rock cores 215 while drilling the well 205, smaller “sidewall rock cores” may be obtained after drilling a portion or all of the well 205. A sidewall rock coring system (not shown) may be lowered by wireline into the well 205. When deployed, the sidewall rock coring system presses or clamps itself against the wall of the well 205 and a sidewall rock core is obtained either by drilling into the wall of the well 205 with a hollow coring bit or by firing a hollow bullet into the wall of the well 205 using an explosive charge. More than 50 such sidewall rock cores may be obtained during a single deployment of a sidewall rock coring system into the well 205. Hereinafter, the term “rock coring system” is used to describe the rock coring system 200 as illustrated in FIG. 2 or the sidewall rock coring system. Further, the term “rock cores” is used to describe the rock cores 215 obtained using either the rock coring system 200 as illustrated in FIG. 2 or the sidewall rock coring system.

In general, the rock cores 215 may be collected along any portion of the well 205. However, in the context of this disclosure, rock cores 215 are rock cores collected along the well 205 that intersects source rock and/or reservoir rock within a stratum 245. As such, rock cores 215 contain kerogen and/or hydrocarbons of various thermal maturities.

Under ideal circumstances, each rock core 215 is recovered as a single, continuous, intact cylinder of the source rock and/or reservoir rock. However, frequently rock cores 215 take the form of several shorter cylindrical segments separated by breaks. The breaks may be a consequence of stresses experienced by the rock cores 215 during coring or may be caused by pre-existing vugs, channels, and/or fractures within strata 245 within the subterranean region 210.

In general, each rock core 215 may be up to 15 centimeters in diameter and approximately ten meters long. To prepare a rock core 215 for pyrolysis testing in a laboratory setting, each rock core 215 may be cut into multiple rock samples (e.g., core plugs). Each rock sample may be in the shape of a cylinder (e.g., disc) or cuboid where each dimension is on the order of centimeters, though other shapes and dimensions may be used. Further, each rock sample may be cut along a particular axis of the well 205, such as parallel or perpendicular to the well 205. Further still, each rock sample may be cut and/or ground into multiple sub-samples. Each sub-sample may be on the order of milligrams. In some embodiments, a sub-sample may contain greater than 1% total organic carbon (TOC). In other embodiments, a sub-sample may be isolated kerogen.

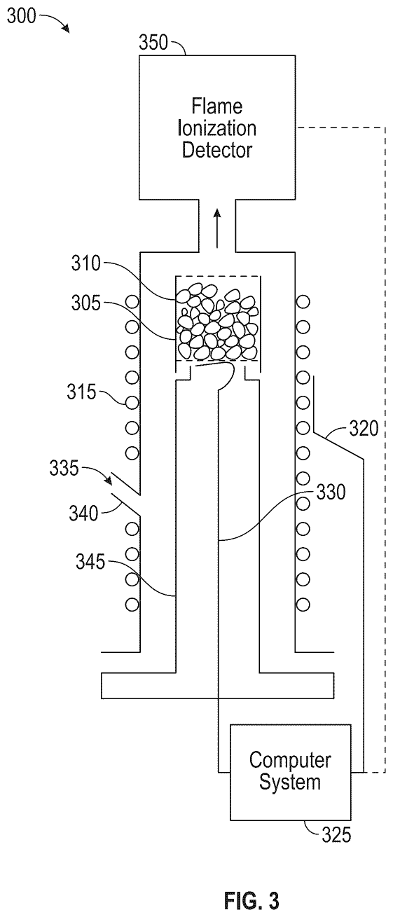

In the laboratory setting, pyrolysis testing of a sub-sample may be performed using a pyrolysis system. FIG. 3 illustrates a pyrolysis system 300 in accordance with one or more embodiments. Pyrolysis may be the process of thermally decomposing and analyzing a sub-sample. The pyrolysis system 300 may be an open or closed system. For example, the pyrolysis system 300 may perform a pyrolysis test in an inert atmosphere (i.e., in the absence of oxygen). Pyrolysis and/or pyrolysis systems 300 may be referred to as Rock-Eval (e.g., Rock-Eval 6 and Rock-Eval 7), SR Analyzer, HAWK, POPI-TOC, and Pyromat.

While FIG. 3 illustrates pyrolysis, features and processes illustrated in and discussed relative to FIG. 3 are not meant to limit the present disclosure. Further, the discussion of the pyrolysis system 300 herein focuses on the use of the pyrolysis system 300 in the context of this disclosure. A person of ordinary skill in the art will appreciate that the pyrolysis system 300 may include other features and other functions not discussed herein configured to further characterize the sub-sample by thermally decomposing the sub-sample.

In some embodiments, as illustrated in FIG. 3, the sub-sample may take the form of a ground sub-sample 305. The ground sub-sample 305 may be loaded into a crucible 310. The crucible 310 may be loaded into a furnace 315. During a pyrolysis test, the furnace 315 may heat the ground sub-sample 305 based on a prescribed heating rate. Common prescribed heating rates may range from 0.5 to 50 degree Celsius per minute (° C./min). As such, common prescribed heating rates may include, but are not limited to, 1° C./min, 3° C./min, 10° C./min, 30° C./min, and 50° C./min. To ensure the prescribed heating rate is maintained during pyrolysis testing, a first thermocouple 320 may measure the temperature of the furnace 315 during pyrolysis testing based on a first pre-determined sampling rate. The temperatures measured by the first thermocouple 320 may be sent to a computer system 325 to be used as feedback. A second thermocouple 330 may measure the temperature of the ground sub-sample 305 during pyrolysis testing based on a second pre-determined sampling rate. The temperatures measured by the second thermocouple 330 may also be sent to the computer system 325 to be used in conjunction with other pyrolysis measurements following pyrolysis testing. In some embodiments, to ensure the ground sub-sample 305 is maintained in an inert atmosphere during pyrolysis testing, nitrogen 335 or other inert gas may be injected into the pyrolysis system 300 via an opening 340.

As the furnace 315 heats the ground sub-sample 305 based on the prescribed heating rate during pyrolysis testing, constituents of the ground sub-sample 305 volatilize or pyrolyze at discrete times and discrete temperatures as pyrolysate. A piston 345 causes the volatilized or pyrolyzed constituents to travel to a flame ionization detector (FID) 350 where the volatilized or pyrolyzed constituents are detected. The detected FID signals may be converted to electrical signals and transferred to and stored on the computer system 325. The computer system 325 may determine the reaction rate k associated to each temperature of the ground sub-sample 305 (hereinafter “pyrolysis data” or “reaction data”) based, at least in part, on the electrical signals.

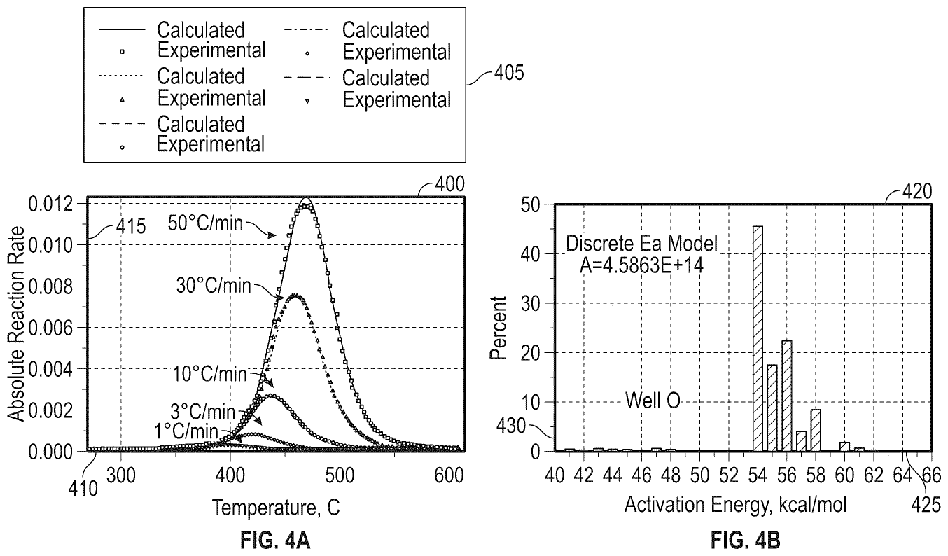

FIG. 4A displays pyrolysis data 400 in accordance with one or more embodiments. FIG. 4A specifically displays pyrolysis data 400 for five ground sub-samples 305 of a rock sample. Each ground sub-sample 305 is pyrolyzed at a unique prescribed heating rate as shown by the points in FIG. 4A labeled as “experimental” in the key 405. The abscissa 410 displays the temperature T of each ground sub-sample 305 during pyrolysis testing in degrees Celsius. The ordinate 415 displays the absolute reaction rate for each temperature T. Note the absolute reaction rate may be the rate of production of pyrolysate from a sample and be controlled by the reaction rate k and the volume of the remaining unpyrolyzed portion of the sample. Further, for a heterogeneous sample, the absolute reaction rate may include contributions from a plurality of materials forming the sample, each with its own reaction rate k.

While FIG. 4A displays pyrolysis data 400 for each of multiple prescribed heating rates, a person of ordinary skill in the art will appreciate that a prescribed heating rate may be prescribed more than once. That is, two ground sub-samples 305 may be pyrolyzed at the same prescribed heating rate during two separate pyrolysis tests. In some embodiments, the smallest and largest prescribed heating rate (e.g., 1° C./min and 50° C./min) may be prescribed twice or once for each of two ground sub-samples 305. A person of ordinary skill in the art will further appreciate that the pyrolysis data 400 may be altered such that the pyrolysis data 400 is free from outliers, smooth, temperature corrected, adjusted or shifted, resampled, and/or normalized without departing from the scope of the disclosure.

The Arrhenius-type model of Equation (1) may be fit to the pyrolysis data 400 associated to each prescribed heating rate as shown by the lines in FIG. 4A labeled as “calculated” in the key 405. The fitting process may be based on a kinetics model such as, but not limited to, a discrete model, Gaussian model, 1^{st} or N^{th} order models, Weibull model, nucleation model, alternate-pathway model, and isoconversional model. The fit of the pyrolysis data 400 for two or more prescribed heating rates may be used to determine a distribution of a kinetic parameter. In some embodiments, the fitting process may be performed within software such as, but not limited to, Kinetics2000™, Kinetics05™ Kinetics2015™, and in-house software.

FIG. 4B displays a distribution of a kinetic parameter value 420 in accordance with one or more embodiments. Specifically, FIG. 4B displays a discrete distribution of activation energies Eₐ. The abscissa 425 displays activation energy Eₐ while the ordinate 430 displays the frequency with which each activation energy is observed as a percentage. In other embodiments, the distribution of the kinetic parameter value 420 may be a continuous Gaussian distribution. The distribution of the kinetic parameter value 420 may be considered complicated as the distribution of the kinetic parameter value 420 is tied to a constant A.

A single scalar value of a weighted kinetic parameter value may be obtained from a distribution of kinetic parameter values, such as that shown in FIG. 4B. The distribution of the activation energy may be simplified to an average value of the activation energy (hereinafter “weighted activation energy”). In some embodiments, the weighted activation energy may be determined using the distribution of the activation energy and a weight function. In some embodiments, the weight function may take the form:

$${E}_{a , W A} = {\sum }_{i = 1}^{n} {E}_{ai} {w}_{i} , \qquad Equation ( 2 )$$

where Eₐ_{ᵢ} is the activation energy at each discrete position along the abscissa 425, wᵢ is the weight associated to each Eₐ_{ᵢ}, and E_{a,WA} is the weighted activation energy. In some embodiments, the weight wᵢ may be a normalized percent, where percent is displayed along the ordinate 430 in FIG. 4B. In Equation (2), E_{a,WA} may be considered a weighted average value of the distribution of the activation energy. However, a person of ordinary skill in the art will appreciate that the weighted activation energy may take alternative forms that may rely on a different weight function or no weight function such as, but not limited to, the maximum Eₐ_{ᵢ} or the median Eₐ_{ᵢ} of the distribution of the kinetic parameter value 420.

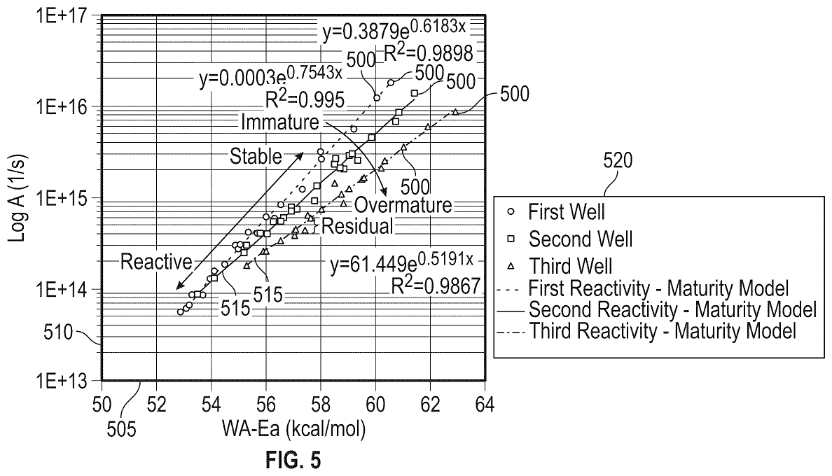

The process of pyrolysis testing two or more ground sub-samples 305 to ultimately determine a weighted kinetic parameter for a rock sample may be repeated for multiple rock samples obtained from each of multiple wells 205 within a subterranean region 210. For example, FIG. 5 displays the weighted kinetic parameter, specifically the weighted-average activation energy, relative to the logarithm of the pre-exponential frequency factor A (hereinafter “first points” 500) for rock samples collected from three wells 205. The abscissa 505 displays the kinetic parameter. The ordinate 510 displays the logarithm of the pre-exponential frequency factor A. In some embodiments, the first points 500 associated to each well 205 may be fit to a reactivity-maturity model 515 as shown by the key 520 in FIG. 5. The fit reactivity-maturity model 515 may be one or more models that reasonably fit the first points 500 associated to each well 205. For example, while FIG. 5 fits the first points 500 associated to each well 205 to one natural exponential function, the first points 500 may be fit to an exponential function or linear function. Further, each subset of first points 500 associated to each well 205 may be fit to a reactivity-maturity model 515. Fitting each subset of first points 500 associated to each well 205 may be performed when there is an indication that the thermal maturity of the stratum 245 surrounding a well 205 is increasing by depth. Indications may be based on vitrinite reflectance R₀ (%) or the peak temperature of pyrolysate, Tₘₐₓ, during the standard pyrolysis on a ground sub-sample 305, for example.

In some embodiments, each fit reactivity-maturity model 515 may be used to remove outliers. For example, a residual between a first point 500 and a fit reactivity-maturity model 515 that is above a threshold may indicate that the first point 500, and thus weighted activation energy associated with that first point 500, is an outliner and should be removed.

In some embodiments, each fit reactivity-maturity model 515 may be an indicator of relative thermal reactivity (hereinafter also “thermal reactivity,” “reactivity,” and “reactive”) as illustrated by the straight arrow in FIG. 5. In the context of this disclosure, thermal reactivity may be defined as the ability of a source rock and/or reservoir rock to resist breaking down when heated and stressed. The greater the thermal reactivity, the quicker the source rock and/or reservoir rock breaks down. Relative thermal reactivity may be antonymous to relative thermal stability (hereinafter also “thermal stability,” “stability,” and “stable”).

In some embodiments, the fit reactivity-maturity models 515 may be indicators of relative thermal maturity (hereinafter also “thermal maturity,” “maturity,” and “mature”) as illustrated by the curved arrow in FIG. 5. In the context of this disclosure, thermal maturity may be defined as the degree of heating of source rock and/or reservoir rock in the process of transforming kerogen into hydrocarbons.

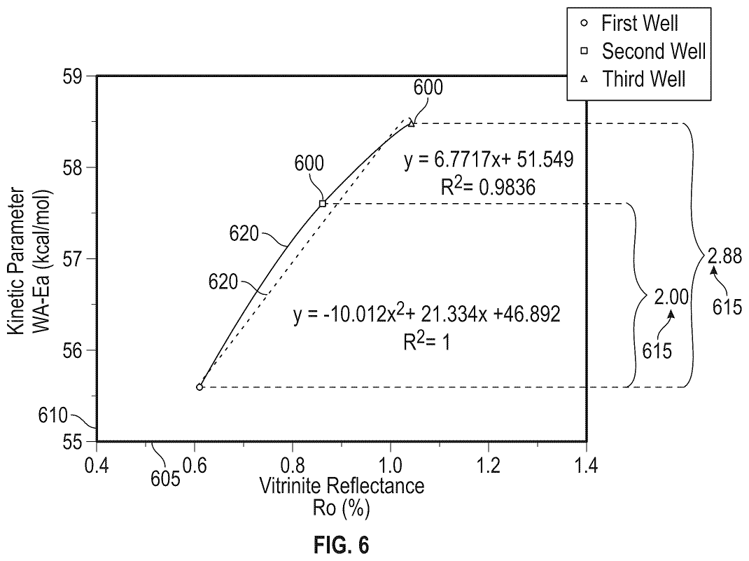

FIG. 6 displays the average of the weighted activation energy value for each well 205 relative to vitrinite reflectance (hereinafter “third points” 600). The abscissa 605 displays vitrinite reflectance Ro. The ordinate 610 displays the average of the weighted activation energy. Vitrinite reflectance is a common measure of thermal maturity. The difference 615 between the average of the weighted activation energy value associated to each of the wells 205 and the average of the weighted activation energy value of the well 205 that penetrates the most thermally-immature hydrocarbons relative to the other wells 205 (hereinafter “first well”) may provide a measure of relative thermal maturity. For example, as illustrated in FIG. 6, the difference 615 between the average of the weighted activation energy value of the first well and the second well is 2.00. The difference 615 between the average of the weighted activation energy value of the first well and the third well is 2.88. As such, in this example, the first well penetrates thermally-immature hydrocarbons; the second well, moderately-mature hydrocarbons; and the third well, mature hydrocarbons. However, a person of ordinary skill in the art will appreciate that the thermal maturity associated with the second well and third well is relative to the first well. As such, if an additional well penetrates thermally-immature hydrocarbons relative to the first well, the additional well is then considered the first well and the differences 615 determined from the new first well.

One or more models 620 may be fit to the three points 600 as illustrated in FIG. 6. The one or more models 620 may be any model that reasonably fits the third points 600. The one or more models 620 may be considered a correlation between the average of the weighted activation energy value for each well 205 and vitrinite reflectance. In some embodiments, a model 620 may be used to estimate the average of the weighted activation energy value based on vitrinite reflectance or some other measure of thermal maturity.

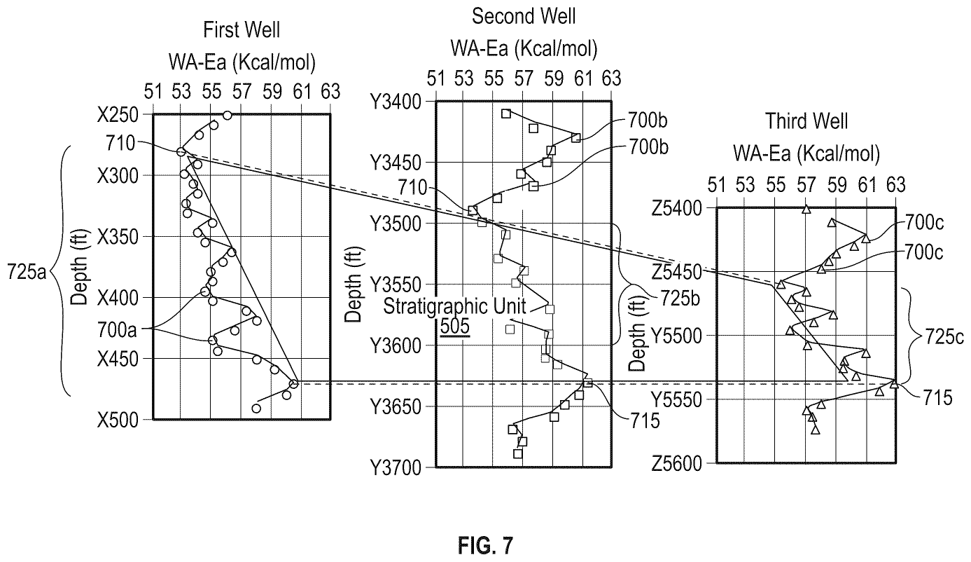

The difference 615 may be used to adjust or calibrate the values of the kinematic parameter within and that define the boundaries of the correlated stratigraphic unit 705 as illustrated in FIG. 7.

FIG. 7 displays values of a weighted activation energy. Specifically, FIG. 7 displays values of a weighted-average activation energy 700a-c as a function of sample depth for three wells. Here, depth may be the depth along each well that each rock sample is obtained from or may be the true vertical depth from which each rock sample is obtained from. Specifically, for example, the data points 700a on the left are associated with a first well, the data points 700b in the middle are associated with a second well, and the data points 700c on the right are associated with a third well.

The data points associated with each well may be used to identify chemostratigraphic segment in each well, such as the chemostratigraphic segment 725a in the first well, the chemostratigraphic segment 725b in the second well, and the chemostratigraphic segment 725c in the third well. The chemostratigraphic segment 725a in the first well may span a first range of depths. Similarly, the chemostratigraphic segment 725b in the second well may span a second range of depths and the chemostratigraphic segment 725c in the third well may span a third range of depths. A correlated stratigraphic unit 705 may be determined using two or more chemostratigraphic segments. This process may be referred to as “chemostratigraphic correlation.” The correlated stratigraphic unit 705 may be associated with a stratum 245 within the subterranean region 210. In some embodiments, the absolute maximum weighted activation energy value 710 and the absolute minimum weighted activation energy value 715 relative to each well 205 may define the boundaries of the correlated stratigraphic unit 705 as illustrated in FIG. 7. In other embodiments, a relative maximum weighted activation energy value and relative minimum weighted activation energy value relative to each well 205 may define the boundaries of the correlated stratigraphic unit 705. However, a person of ordinary skill in the art will appreciate that still other methods may be used to identify the correlated stratigraphic unit 705.

The weighted activation energy values within and at the boundaries of the correlated stratigraphic unit 705 may be used to quantify the relative thermal maturity of the hydrocarbons within the associated stratum 245 that each well 205 penetrates. To do so, the average of the weighted activation energy values within and on the boundaries of the correlated stratigraphic unit 705 for each well 205 may be determined.

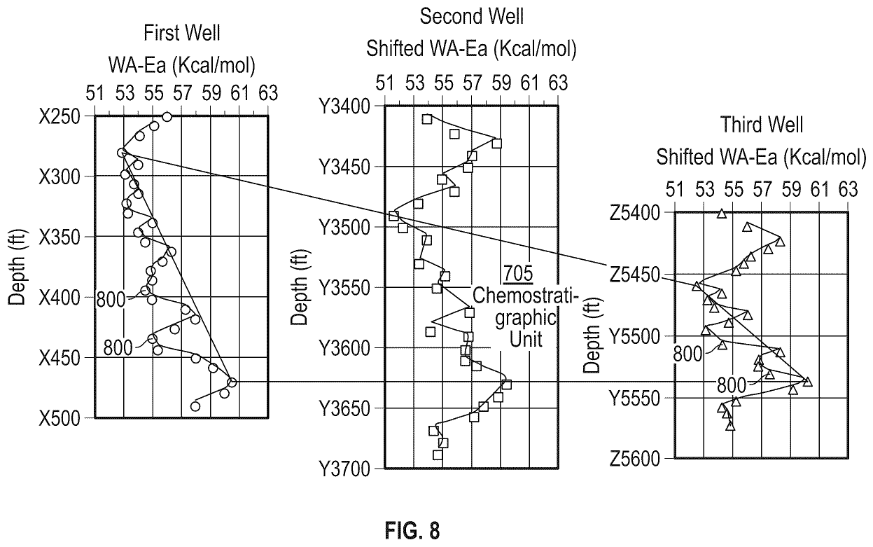

FIG. 8 displays the maturity-corrected kinetic parameter value relative to depth (hereinafter “fourth points” 800). In these embodiments, because the fourth points 800 associated with the first well on the left in FIG. 8 penetrates the most immature hydrocarbons relative to the second and third well, the fourth points 800 relative to the first well are the same as the second points 600 displayed in FIG. 6. However, the fourth points 800 associated with the second well in the middle of FIG. 8 are now adjusted by subtracting the difference 615 between the first well and second well (e.g., 2.00 per FIG. 7) from the weighted activation energy values. Further, the fourth points 800 associated with the third well on the right in FIG. 8 are now adjusted by subtracting the difference 615 between the first well and third well (e.g., 2.88 per FIG. 6) from the weighted activation energy values.

A unique organofacies may now be assigned to the stratum 245 associated to the correlated stratigraphic unit 705 that surrounds each well 205 based, at least in part, on the maturity-corrected kinetic parameter values. In some embodiments, the same unique organofacies may be assigned to the stratum 245 that surrounds each well 205. In these embodiments, one organofacies may be assigned to the stratum 245. Further, in some embodiments, a unique organofacies may be assigned to the stratum 245 associated to the correlated stratigraphic unit 705 that surrounds each well 205 based, at least in part, on the weighted activation energy values.

The assigned organofacies may be based on a more detailed organofacies classification system than the one displayed in FIG. 1. In the detailed organofacies classification system, the maturity-corrected kinetic parameter value may be considered a new characterization measure. In some embodiments, the maturity-corrected kinetic parameter may further define organofacies A, B, C, D/E, and F, may define sub-organofacies within organofacies A, B, C, D/E, and/or F, and/or may define additional organofacies beyond organofacies A, B, C, D/E, and F.

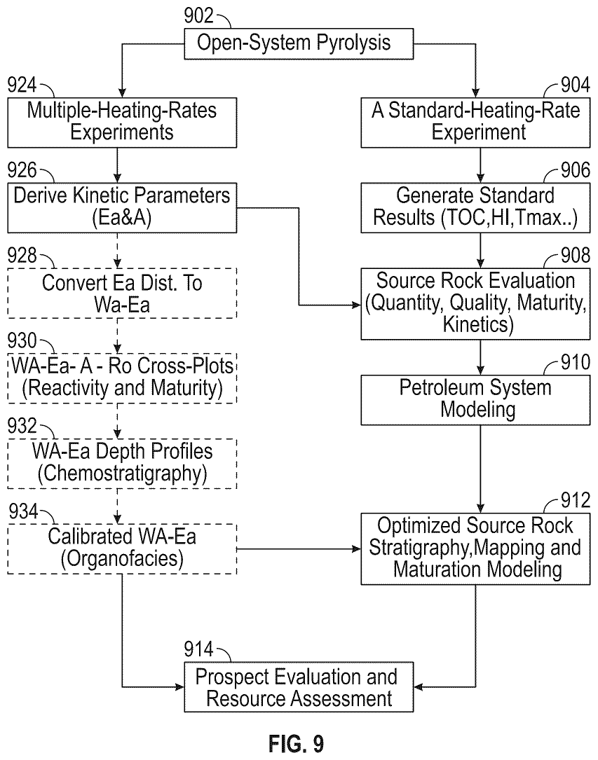

FIG. 9 shows a flowchart in accordance with one or more embodiments. Specifically, FIG. 9 relates to embodiments where the kinetic parameter values are an activation energy E_{A} and a pre-exponential A (see the Arrhenius-type model, Equation (1)) and the weighted kinetic parameter value is a weighted-average activation energy average, E_{a,WA}. FIG. 9 shows two paths through the workflow: a standard workflow (right column) and a novel workflow (left column).

In step 902, open-system pyrolysis may be used to derive pyrolysis data from a plurality of rock samples. In the standard workflow, a single heating rate experiment may be performed 904. The standard results 906 generated may include values for total organic contents (TOC), hydrocarbon index (HI), and the temperature at the pyrolysate peak (Tₘₐₓ). In step 908, the quality of the source rock may be evaluated including, without limitation, the maturity and kinetics of the source rock. The resulting source rock evaluation parameters may then be included in basin modeling or petroleum system modeling 910 to predict the evolution over geological time of the hydrocarbon reservoir and the surrounding strata above and below the hydrocarbon reservoir. The results of the petroleum system modeling 910 may be used to map the source rock stratigraphy across the hydrocarbon reservoir and improve, update, or optimize the understanding of the maturation level of the source rock as a function of depth and horizontal position. These maps may be used to determine parameters such as estimated ultimate recovery from the hydrocarbon reservoir as well as determine information such as preferred recovery strategies including advantageous drilling targets within the hydrocarbon reservoir. This collection of uses of the stratigraphy and maturity maps are frequently termed “Prospect Evaluation” and “Resource Assessment” 914.

In a variant of the standard workflow, the open-system pyrolysis equipment may be used to perform pyrolysis on a rock sample, or portions of a rock sample (e.g., a ground sub-sample 305), at multiple heating rates 924. The results from such multiple heating rate experiments may be used to determine kinetic parameters 926 such as activation energies and pre-exponential factors. In standard workflows, the kinetic parameters 926 may be used in the source rock evaluation in step 908 to supplement TOC, HI, and Tₘₐₓ, among others.

In embodiments of the novel workflow, the distribution of activation energies may be converted into a weighted-average activation energy E_{a,WA} 928. In step 932 cross-plots of weighted average activation energy E_{a,WA} and vitrinite reflectance R₀ may be used to determine the reactivity and maturity of the rock sample. Depth profiles (well logs) of the weighted-average activation energy may be formed in step 932 and chemostratigraphy performed to associate strata with a pattern of characteristic weighted-average activation energies in different wells 205 with one another and estimate the location of the associated strata in the regions between wells 205.

In step 934, the maturity level determined in step 930 may be combined with the weighted-average activation energy determined in step 932 to produced calibrated weighted-average activation energy values that are corrected for the maturity level of the rock samples. These calibrated weighted-average activation energy values may be used to determine the organofacies to which each rock sample belongs. The organofacies classification may be used directly for prospect evaluation and resource assessment 914 and/or may be combined with the conventionally obtained mapping of stratigraphy and maturity maps in step 912.

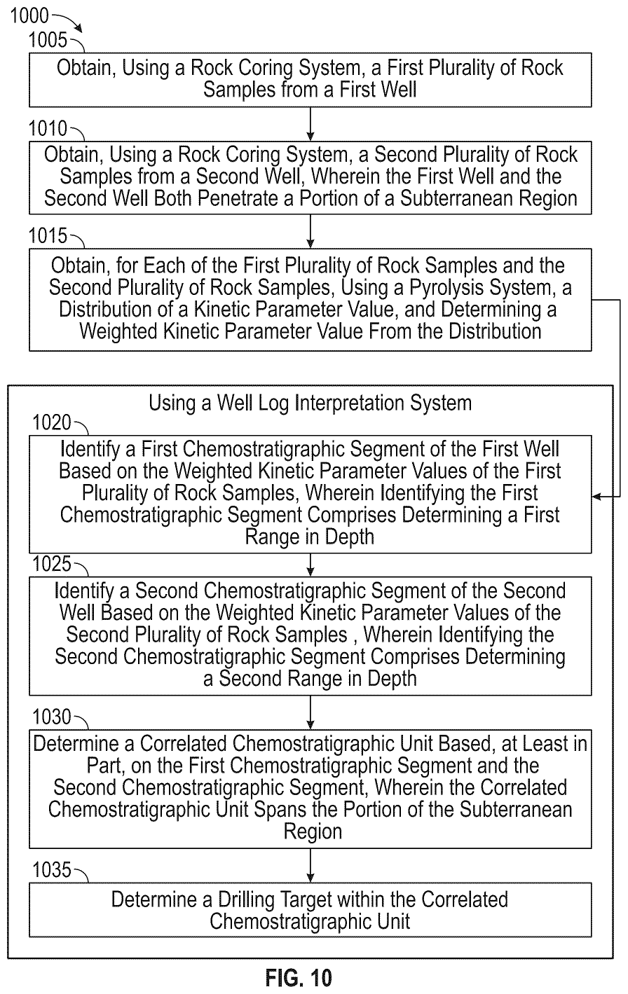

FIG. 10 shows a flowchart 1000 representing a process in accordance with one or more embodiments. In step 1005, a first plurality of rock samples may be obtained from a first well using a rock coring system 200. Similarly, in step 1010, a second plurality of rock samples may be obtained from a second well using the rock coring system 200. Both the first well and the second well penetrate a portion of a subterranean region 210. The first plurality of rock samples and/or the second plurality of samples may include rock samples of a hydrocarbon source rock. In some embodiments, the first kinetic parameter value may be an activation energy value.

Steps 1020, 1025, 1030 and 1035 may be performed using a well log interpretation system.

In step 1015, for each of the first plurality of rock samples and the second plurality of rock samples, a distribution of a kinetic parameter value 420 may be obtained using a pyrolysis system 300. A weighted kinetic parameter value may be determined from the distribution of the kinetic parameter value 420. In some embodiments, the weighted kinetic parameter value may include a weighted-average activation energy value.

In step 1020, a first chemostratigraphic segment of the first well may be identified based on the weighted kinetic parameter values of the first plurality of rock samples. Identifying the first chemostratigraphic segment may include determining a first range in depth spanned by the first chemostratigraphic segment. In some embodiments, the range in depth may be determined by locating maximum and/or minimum values of the weighted kinetic parameter value, while in other embodiments, the range in depth may be determined by a characteristic pattern of variation with depth, such as an oscillation with a characteristic spatial period, of the weighted kinetic parameter value.

In step 1025, a second chemostratigraphic segment of the second well based on the weighted kinetic parameter values of the second plurality of rock samples, where identifying the second chemostratigraphic segment including determining a second range in depth. The second chemostratigraphic segment of the second well may be identified using the same criteria as used to identify the first chemostratigraphic segment in the first well.

In step 1030, a correlated stratigraphic unit 705 based, at least in part, on the first chemostratigraphic segment and the second chemostratigraphic segment may be identified. The correlated stratigraphic unit may span the portion of the subterranean region between the first well and the second well.

In step 1035, a drilling target may be determined within the correlated stratigraphic unit. Determining the drilling target within the correlated stratigraphic unit may include determining a first thermal maturity shift for the first chemostratigraphic segment and a second thermal maturity shift for the second chemostratigraphic segment. Determining the drilling target may also include determining a first maturity-corrected kinetic parameter value for the first chemostratigraphic segment based, at least in part, on the first thermal maturity shift and a second maturity-corrected kinetic parameter value for the second chemostratigraphic segment based, at least in part, on the second thermal maturity shift. Determining the drilling target may further include determining an organofacies based, at least in part, on the first maturity-corrected kinetic parameter value and the second maturity-corrected kinetic parameter value. Determining the organofacies may include obtaining a measured vitrinite reflectance for at least one of the rock samples.

The method may further include obtaining, using a petroleum system modeling system, a petroleum system model (PSM) configured to model hydrocarbon generation and accumulation within the subterranean region, inputting the organofacies into the PSM, and predicting the hydrocarbon generation, retention, and expulsion in the source rock, at least in part, on the organofacies. The method may still further include determining a current location of a prospect of hydrocarbon accumulation in conventional reservoirs and unconventional reservoirs, such as shale reservoirs, at least in part, on the predicted hydrocarbon generation and expulsion, and planning, using a wellbore planning system, a planned wellbore path that penetrates the mature portion of the hydrocarbon prospect based, at least in part, on the current location. A wellbore may be drilled, using a drilling system, guided by the planned wellbore path.

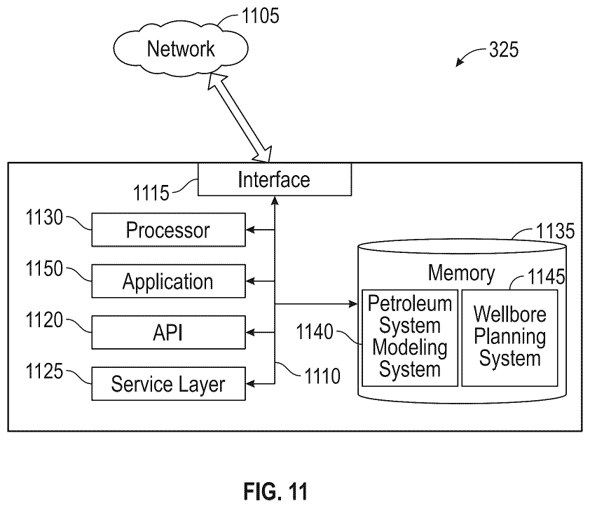

FIG. 11 depicts a block diagram of a computer system 325 used to provide computational functionalities associated with described algorithms, methods, functions, processes, flows, and procedures as described in this disclosure, according to one or more embodiments. Specifically, the computer system 325 may be configured to perform steps 1020, 1025, 1030, and 1035 as previously described relative to FIG. 10. The illustrated computer system 325 is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer system 325 may include a computer that includes an input device, such as a keypad, keyboard, touch screen, or other device that can accept user information, and an output device that conveys information associated with the operation of the computer system 325, including digital data, visual, or audio information (or a combination of information), or a GUI.

The computer system 325 can serve in a role as a client, network component, a server, a database or other persistency, or any other component (or a combination of roles) of a computer system for performing the subject matter described in the instant disclosure. The illustrated computer system 325 is communicably coupled with a network 1105. In some implementations, one or more components of the computer system 325 may be configured to operate within environments, including cloud-computing-based, local, global, or other environment (or a combination of environments).

At a high level, the computer system 325 is an electronic computing device operable to receive, transmit, process, store, or manage data and information associated with the described subject matter. According to some implementations, the computer system 325 may also include or be communicably coupled with an application server, e-mail server, web server, caching server, streaming data server, business intelligence (BI) server, or other server (or a combination of servers).

The computer system 325 can receive requests over network 1105 from a client application (for example, executing on another computer system 325) and responding to the received requests by processing the said requests in an appropriate software application. In addition, requests may also be sent to the computer system 325 from internal users (for example, from a command console or by other appropriate access method), external or third-parties, other automated applications, as well as any other appropriate entities, individuals, systems, or computers.

Each of the components of the computer system 325 can communicate using a system bus 1110. In some implementations, any or all of the components of the computer system 325, both hardware or software (or a combination of hardware and software), may interface with each other or the interface 1115 (or a combination of both) over the system bus 1110 using an application programming interface (API) 1120 or a service layer 1125 (or a combination of the API 1120 and service layer 1125. The API 1120 may include specifications for routines, data structures, and object classes. The API 1120 may be either computer-language independent or dependent and refers to a complete interface, a single function, or even a set of APIs. The service layer 1125 provides software services to the computer system 325 or other components (whether or not illustrated) that are communicably coupled to the computer system 325. The functionality of the computer system 325 may be accessible for all service consumers using this service layer. Software services, such as those provided by the service layer 1125, provide reusable, defined business functionalities through a defined interface. For example, the interface may be software written in JAVA, C++, or other suitable language providing data in extensible markup language (XML) format or another suitable format. While illustrated as an integrated component of the computer system 325, alternative implementations may illustrate the API 1120 or the service layer 1125 as stand-alone components in relation to other components of the computer system 325 or other components (whether or not illustrated) that are communicably coupled to the computer system 325. Moreover, any or all parts of the API 1120 or the service layer 1125 may be implemented as child or sub-modules of another software module, enterprise application, or hardware module without departing from the scope of this disclosure.

The computer system 325 includes an interface 1115. Although illustrated as a single interface 1115 in FIG. 11, two or more interfaces 1115 may be used according to particular needs, desires, or particular implementations of the computer system 325. The interface 1115 is used by the computer system 325 for communicating with other systems in a distributed environment that are connected to the network 1105. Generally, the interface 1115 includes logic encoded in software or hardware (or a combination of software and hardware) and operable to communicate with the network 1105. More specifically, the interface 1115 may include software supporting one or more communication protocols associated with communications such that the network 1105 or interface's hardware is operable to communicate physical signals within and outside of the illustrated computer system 325.

The computer system 325 includes at least one computer processor 1130. Although illustrated as a single computer processor 1130 in FIG. 11, two or more processors may be used according to particular needs, desires, or particular implementations of the computer system 325. Generally, the computer processor 1130 executes instructions and manipulates data to perform the operations of the computer system 325 and any algorithms, methods, functions, processes, flows, and procedures as described in the instant disclosure.

The computer system 325 also includes a memory 1135 that stores various data for the computer system 325 or other components (or a combination of both) that can be connected to the network 1105. In the context of this disclosure, the memory 1135 may store the pyrolysis data 400 as well as a petroleum system modeling system 1140 and wellbore planning system 1145 in the form of software as shown in FIG. 11. In some embodiments, the petroleum system modeling system 1140 may be configured to determine the petroleum system model. In some embodiments, the wellbore planning system 1145 may be configured to plan the wellbore path. Although illustrated as a single memory 1135 in FIG. 11, two or more memories may be used according to particular needs, desires, or particular implementations of the computer system 325 and the described functionality. While memory 1135 is illustrated as an integral component of the computer system 325, in alternative implementations, memory 1135 can be external to the computer system 325.

The application 1150 is an algorithmic software engine providing functionality according to particular needs, desires, or particular implementations of the computer system 325, particularly with respect to functionality described in this disclosure. For example, application 1150 can serve as one or more components, modules, applications, etc. Further, although illustrated as a single application 1150, the application 1150 may be implemented as multiple applications 1150 on the computer system 325. In addition, although illustrated as integral to the computer system 325, in alternative implementations, the application 1150 can be external to the computer system 325.

There may be any number of computer systems 325 associated with, or external to, a computer system containing a computer system 325, wherein each computer system 325 communicates over network 1105. Further, the term “client,” “user,” and other appropriate terminology may be used interchangeably as appropriate without departing from the scope of this disclosure. Moreover, this disclosure contemplates that many users may use one computer system 325, or that one user may use multiple computer systems 325.

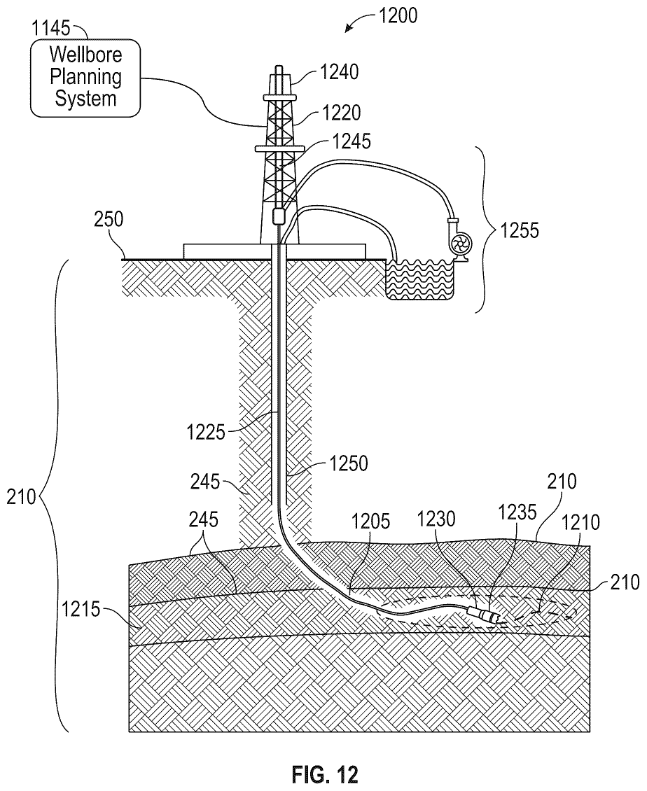

FIG. 12 illustrates a drilling system 1200 in accordance with one or more embodiments. A wellbore 1205 may be drilled, using the drilling system 1200, guided by the wellbore path 1210 to penetrate the mature hydrocarbon reservoir 1215 within the subterranean region 210. Although the drilling system 1200 shown in FIG. 12 is used to drill the wellbore 1205 on land, the drilling system 1200 may be a marine wellbore drilling system. Further, although the drilling system 1200 shown in FIG. 12 is used to drill a new wellbore 1205, the wellbore 1205 being drilled may be a sidetrack wellbore or an offset wellbore. As such, the example of the drilling system 1200 shown in FIG. 12 is not meant to limit the present disclosure.

As shown in FIG. 12, the wellbore 1205 may be drilled using a drill rig that may be situated on a land drill site, an offshore platform, such as a jack-up rig, a semi-submersible, or a drill ship. The drill rig may be equipped with a hoisting system, such as a derrick 1220, which can raise or lower the drillstring 1225 and other tools required to drill the wellbore 1205. The drillstring 1225 may include one or more drill pipes connected to form conduit and a bottom hole assembly 1230 (BHA) disposed at the distal end of the drillstring 1225. The BHA 1230 may include a drill bit 1235 to cut into strata 245. The BHA 1230 may further include measurement tools, such as a measurement-while-drilling (MWD) tool and logging-while-drilling (LWD) tool. MWD tools may include sensors and hardware to measure downhole drilling parameters, such as the azimuth and inclination of the drill bit 1235, the weight-on-bit, and the torque. The LWD measurements may include sensors, such as resistivity, gamma ray, and neutron density sensors, to characterize the strata 245 surrounding the wellbore 1205. Both MWD and LWD measurements may be transmitted to the surface of the earth 250 using any suitable telemetry system known in the art, such as a mud-pulse or by wired-drill pipe.

To start drilling, or “spudding in,” the wellbore 1205, the hoisting system lowers the drillstring 1225 suspended from the derrick 1220 of the drill rig towards the planned surface location of the wellbore 1205. An engine, such as a diesel engine, may be used to supply power to the top drive 1240 to rotate the drillstring 1225 via the drive shaft 1245. The weight of the drillstring 1225 combined with the rotational motion enables the drill bit 1235 to bore the wellbore 1205.

The near-surface strata 245 of the subterranean region 210 is typically made up of loose or soft sediment or rock, so large diameter casing 1250 (e.g., “base pipe” or “conductor casing”) is often put in place while drilling to stabilize and isolate the wellbore 1205. At the top of the base pipe is the wellhead, which serves to provide pressure control through a series of spools, valves, or adapters (not shown). Once near-surface drilling has begun, water or drill fluid may be used to force the base pipe into place using a pumping system until the wellhead is situated just above the surface of the earth 250.

Drilling may continue without any casing 1250 once deeper or more compact strata 245 is reached. While drilling, a drilling mud system 1255 may pump drilling mud from a mud tank on the surface of the earth 250 through the drill pipe. Drilling mud serves various purposes, including pressure equalization, removal of rock cuttings, and drill bit cooling and lubrication.

At planned depth intervals, drilling may be paused and the drillstring 1225 withdrawn from the wellbore 1205. Sections of casing 1250 may be connected, inserted, and cemented into the wellbore 1205. Casing string may be cemented in place by pumping cement and mud, separated by a “cementing plug,” from the surface of the earth 250 through the drill pipe. The cementing plug and drilling mud force the cement through the drill pipe and into the annular space between the casing 1250 and the wall of the wellbore 1205. Once the cement cures, drilling may recommence. The drilling process is often performed in several stages. Therefore, the drilling and casing cycle may be repeated more than once, depending on the depth of the wellbore 1205 and the pressure on the walls of the wellbore 1205 from surrounding strata 245.

Due to the high pressures experienced by deep wellbores 1205, a blowout preventer (BOP) may be installed at the wellhead to protect the rig and environment from unplanned oil or gas releases. As the wellbore 1205 becomes deeper, both successively smaller drill bits 1235 and casing 1250 may be used. Drilling deviated or horizontal wellbores 1205 may require specialized drill bits 1235 or drill assemblies.

The drilling system 1200 may be disposed at and communicate with other systems in the wellbore environment. The drilling system 1200 may control at least a portion of a drilling operation by providing controls to various components of the drilling operation. In one or more embodiments, the system may receive data from one or more sensors arranged to measure controllable parameters of the drilling operation. As a non-limiting example, sensors may be arranged to measure weight-on-bit, drill rotational speed (RPM), flow rate of the mud pumps (GPM), and rate of penetration of the drilling operation (ROP). Each sensor may be positioned or configured to measure a desired physical stimulus. Drilling may be considered complete when a drilling target with the mature hydrocarbon reservoir 1215 is reached or the presence of mature hydrocarbons is established.

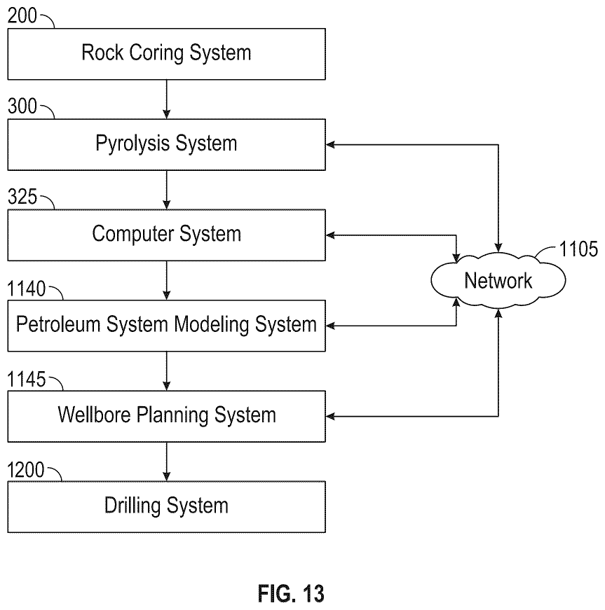

Turning to FIG. 13, FIG. 13 illustrates a systems flowchart in accordance with one or more embodiments. The rock coring system 200 as described relative to FIG. 2 may be configured to obtain the rock samples initially in the form of rock cores 215. The rock cores 215 may be transported to and stored in a laboratory setting. Each rock core 215 may be cut into multiple rock samples. Each rock sample may be ground into multiple ground sub-samples 305.

In the laboratory setting, each ground sub-sample 305 may be pyrolyzed using a pyrolysis system 300 as described relative to FIG. 3. The pyrolysis system 300 may be configured to generate pyrolysis data 400. The pyrolysis data 400 may be transferred to and stored on a computer system 325.

In some embodiments, the computer system 325 may be configured to determine a distribution of a kinetic parameter value 420 from the pyrolysis data 400. The computer system 325 may be configured to perform steps 1020, 1025, 1030, and 1035 as described relative to FIG. 10 to ultimately assign an organofacies to a stratum 245 that surrounds each well 205.

One or more organofacies may be input into a petroleum system model built using a petroleum system modeling system 1140. In some embodiments, the petroleum system modeling system 1140 may be stored in the form of software on the memory 1135 of the computer system 325 as illustrated in FIG. 11. The petroleum system modeling system 1140 may be configured to predict the evolution of the subterranean region 210 based, at least in part, on the one or more organofacies. In some embodiments, the petroleum system modeling system 1140 may be further configured to determine a current location of a mature hydrocarbon reservoir 1215 within the subterranean region 210 based, at least in part, on the predicted evolution of the subterranean region 210.

In some embodiments, a wellbore planning system 1145 may be configured to plan a wellbore path 1210 based, at least in part, on the current location of the mature hydrocarbon reservoir 1215. In some embodiments, the wellbore planning system 1145 may be stored on a memory 1135 of the computer system 325 as illustrated in FIG. 10.

The planned wellbore path 1210 may be loaded into the drilling system 1200 discussed relative to FIG. 12. The drilling system 1200 may be configured to drill the wellbore 1205 within the subterranean region 210 guided by the planned wellbore path 1210. The wellbore 1205 may be a continuation of the previously partially drilled wellbore 1205, a sidetrack wellbore, or an offset wellbore. Following drilling and completion of the wellbore 1205, the wellbore 1205 may be configured to produce mature hydrocarbons from the mature hydrocarbon reservoir 1215 to the surface of the earth 250.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A method comprising:
obtaining, using a rock coring system, a first plurality of hydrocarbon source rock samples from a first well;
obtaining, using the rock coring system, a second plurality of hydrocarbon source rock samples from a second well, wherein the first well and the second well both penetrate a portion of a subterranean region;
for each of the first plurality of hydrocarbon source rock samples and the second plurality of hydrocarbon source rock samples, using a pyrolysis system:
obtaining a set of kinetic parameter values, wherein the set comprises a discrete distribution of activation energies and a common frequency factor, and
determining a weighted average activation energy value from the discrete distribution; and
using a well log interpretation system:
identifying a first chemostratigraphic segment of the first well based on the weighted average activation values of the first plurality of hydrocarbon source rock samples, wherein identifying the first chemostratigraphic segment comprises determining a chemostratigraphic marker spanning a first range in depth,
identifying a second chemostratigraphic segment of the second well based on the weighted average activation values of the second plurality of hydrocarbon source rock samples, wherein identifying the second chemostratigraphic segment comprises determining a second range in depth,
determining a correlated stratigraphic unit based, at least in part, on the first chemostratigraphic segment and the second chemostratigraphic segment, wherein the correlated stratigraphic unit spans the portion of the subterranean region, and
determining a drilling target within the correlated stratigraphic unit.

2. The method of claim 1, wherein determining the drilling target within the correlated stratigraphic unit comprises:
determining a first thermal maturity shift for the first chemostratigraphic segment and a second thermal maturity shift for the second chemostratigraphic segment;
determining a first maturity-corrected activation energy value for the first chemostratigraphic segment based, at least in part, on the first thermal maturity shift;
determining a second maturity-corrected activation energy value for the second chemostratigraphic segment based, at least in part, on the second thermal maturity shift; and
determining an organofacies based, at least in part, on the first maturity-corrected activation energy value and the second maturity-corrected activation energy value.

3. The method of claim 2, further comprising:
obtaining, using a petroleum system modeling system, a petroleum system model (PSM) configured to model hydrocarbon generation and expulsion within the subterranean region;
distinguishing and mapping, using the first maturity-corrected activation energy values and the second maturity-corrected activation energy values, organofacies of a hydrocarbon source rock or a shale reservoir in the PSM; and
predicting the hydrocarbon generation, retention and expulsion in the hydrocarbon source rock or the shale reservoir, at least in part, on the organofacies.

4. The method of claim 3, further comprising:
determining a current location of a mature portion of the shale reservoir based, at least in part, on the predicted hydrocarbon generation, retention and expulsion; and
planning, using a wellbore planning system, a planned wellbore path that penetrates the mature portion of the shale reservoir based, at least in part, on the current location.

5. The method of claim 4, further comprising drilling, using a drilling system, a wellbore guided by the planned wellbore path.

6. The method of claim 2, wherein the first thermal maturity shift is zero.

7. The method of claim 2, wherein the second thermal maturity shift comprises a difference between an average of the weighted activation energy value of the first well and an average of the weighted activation energy value for the second well.

8. The method of claim 1, wherein the chemostratigraphic marker comprises a weighted average activation energy value.

9. The method of claim 1, wherein obtaining the discrete distribution comprises:
generating, using the pyrolysis system, pyrolysis data; and
determining, using an Arrhenius-type model and a kinetics model, the discrete distribution from the pyrolysis data.

10. The method of claim 2, wherein determining the organofacies comprises obtaining a measured vitrinite reflectance and a peak temperature.

11. The method of claim 1, wherein identifying the first chemostratigraphic segment comprises identifying an absolute minimum weighted activation energy value among the weighted average activation values and an absolute maximum weighted activation energy value among the weighted average activation values.

12. A system comprising:
a rock coring system, configured to obtain a first plurality of hydrocarbon source rock samples from a first well and a second plurality of hydrocarbon source rock samples from a second well, wherein the first well and the second well both penetrate a portion of a subterranean region;
a pyrolysis system configured, for each of the first plurality of hydrocarbon source rock samples and the second plurality of hydrocarbon source rock samples, to:
obtain a set of kinetic parameter values, wherein the set comprises a discrete distribution of activation energy and a common frequency factor, and
determine a weighted average activation energy kinetic parameter value from the discrete distribution; and
a well log interpretation system, configured to:
identify a first chemostratigraphic segment of the first well based on the weighted average activation values of the first plurality of hydrocarbon source rock samples, wherein identifying the first chemostratigraphic segment comprises determining a chemostratigraphic marker spanning a first range in depth,
identify a second chemostratigraphic segment of the second well based on the weighted average activation values of the second plurality of hydrocarbon source rock samples, wherein identifying the second chemostratigraphic segment comprises determining a second range in depth,
determine a correlated stratigraphic unit based, at least in part, on the first chemostratigraphic segment and the second chemostratigraphic segment, wherein the correlated stratigraphic unit spans the portion of the subterranean region, and
determine a drilling target within the correlated stratigraphic unit.

13. The system of claim 12, wherein to determine the drilling target within the correlated stratigraphic unit comprises:
determining a first thermal maturity shift for the first chemostratigraphic segment and a second thermal maturity shift for the second chemostratigraphic segment;
determining a first maturity-corrected activation energy value for the first chemostratigraphic segment based, at least in part, on the first thermal maturity shift;
determining a second maturity-corrected activation energy value for the second chemostratigraphic segment based, at least in part, on the second thermal maturity shift; and
determining an organofacies based, at least in part, on the first maturity-corrected activation energy value and the second maturity-corrected activation energy value.

14. The system of claim 13, further comprising a petroleum system modelling system configured to:
obtain a petroleum system model (PSM) configured to model hydrocarbon generation and expulsion within the subterranean region;
distinguish and map, using the first maturity-corrected activation energy values and the second maturity-corrected activation energy values, organofacies of the first plurality of hydrocarbon source rock samples in the PSM; and
predict the hydrocarbon generation, retention and expulsion in the first plurality of hydrocarbon source rock samples, at least in part, on the organofacies.

15. The system of claim 14, wherein the petroleum system modelling system is further configured to determine a current location of a mature portion of a hydrocarbon source rock or shale reservoir based, at least in part, on the predicted hydrocarbon generation, retention and expulsion.

16. The system of claim 15, further comprising:
a wellbore planning system configured to plan a planned wellbore path that penetrates the mature portion of the hydrocarbon source rock or the shale reservoir based, at least in part, on the current location; and
a drilling system configured to drill a wellbore guided by the planned wellbore path.

17. The system of claim 13, wherein the first thermal maturity shift is zero.

18. The system of claim 13, wherein the second thermal maturity shift comprises a difference between an average of the weighted activation energy value of the first well and an average of the weighted activation energy value for the second well.

19. The system of claim 12, wherein to obtain the discrete distribution comprises:
generating, using the pyrolysis system, pyrolysis data; and
determining, using an Arrhenius-type model and a kinetics model, the discrete distribution from the pyrolysis data.

20. The system of claim 13, wherein to determine the organofacies comprises obtaining a measured vitrinite reflectance and a peak temperature.
